(19) United States
(12) Patent Application Publication
(10) Pub. No.: US 2010/0323989 A1
(43) Pub. Date: Dec. 23, 2010

(54) PHARMACEUTICAL COMBINATIONS USEFUL FOR TREATING HCV

(75) Inventors: William E. Delaney, Foster City, CA (US); Hongmei Mo, Palo Alto, CA (US); Weidong Zhong, San Ramon, CA (US)

Correspondence Address: GILEAD SCIENCES INC, 333 LAKESIDE DR, FOSTER CITY, CA, 94404, US

(73) Assignee: Gilead Sciences, Inc., Foster City, CA (US)

(21) Appl. No.: 12/817,392

(22) Filed: Jun. 17, 2010

Related U.S. Application Data
(60) Provisional application No. 61/219,637, filed on Jun. 23, 2009.
(60) Provisional application No. 61/302,418, filed on Feb. 8, 2010.

(51) Int. Cl. A61K 31/675; A61P 31/14

(52) U.S. Cl. 514/82

(57) ABSTRACT

This invention relates to combinations of Compound 1 and Compound 2 which are useful for treating hepatitis C virus infection.

### CROSS-REFERENCE TO RELATED APPLICATIONS

This application is filed under 35 U.S.C. 111(a) claiming the benefit under 35 U.S.C. 119(e) of U.S. Provisional Patent Application No. 61/302,418, filed Feb. 8, 2010 and U.S. Provisional Patent Application No. 61/219,637, filed Jun. 23, 2009 which are herein incorporated by reference in its entirety for all purposes.

### FIELD OF THE INVENTION

This invention relates to combinations of therapeutic molecules useful for treating hepatitis C virus infection.

### BACKGROUND OF THE INVENTION

Hepatitis C is recognized as a chronic viral disease of the liver which is characterized by liver disease. Although drugs targeting the liver are in wide use and have shown effectiveness, toxicity and other side effects have limited their usefulness. Inhibitors of HCV are useful to limit the establishment and progression of infection by HCV as well as in diagnostic assays for HCV.

There is a need for new HCV therapeutic agents.

### SUMMARY OF THE INVENTION

In one aspect, the present invention provides compositions that each include Compound 1 and Compound 2. Compound 1 has the following structure identified as Formula 1:

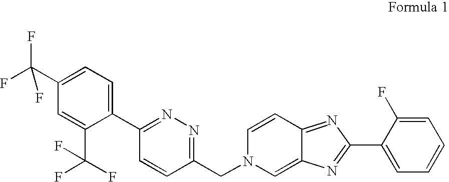

Compound 2 has the following structure identified as Formula 2:

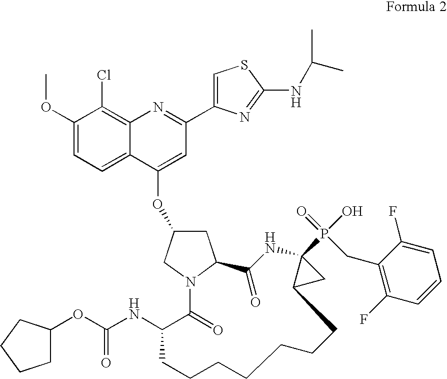

Compounds 1 and 2 both inhibit replication of HCV. While not wishing to be bound by theory, Compound 1 is believed to be an inhibitor of the HCV NS5B polymerase enzyme. Compound 1 is disclosed in PCT/US2007/015553 (WO 2008/005519), which is incorporated herein by reference in its entirety. Again, while not wishing to be bound by theory, Compound 2 is believed to be an inhibitor of the HCV NS3/4A protease enzyme. Compound 2 is disclosed in PCT/US2007/015664, which is incorporated herein by reference in its entirety. The data shown in Example 3 demonstrate the anti-HCV activity of the combination of Compound 1 and Compound 2. Moreover, the data shown in Example 4 show that Compound 2 displays wild-type potency against a panel of known mutants in NS5B that confer resistance to Compound 1. Additionally, the data shown in Examples 5 and 6 show that when Compound 1 and Compound 2 are coadministered to a human being, then Compound 2 increases the drug exposure of Compound 1 as measured by Area Under the Curve (AUC). Thus, in practice, coadministration of Compounds 1 and 2 may permit a smaller, therapeutically effective, amount of Compound 1 to be administered to a human being who is infected with HCV compared to administration of Compound 1 alone. Further, the data shown in Example 7 show a synergistic interaction between Compound 1 and Compound 2 in an in vitro HCV assay.

The data disclosed in Examples 3 thru 7 of the present application therefore suggest the beneficial combination of Compounds 1 and 2 as a treatment for HCV infection.

The compositions of the present invention typically (although not necessarily) include sufficient Compound 1 and Compound 2 to provide a dose of these two compounds that is effective to treat HCV infection when administered to a human being as part of a treatment regime. Thus, for example, in one aspect, the present invention provides pharmaceutical compositions that include Compound 1 and Compound 2 and one or more pharmaceutically acceptable carrier.

Compositions of the present invention can include, for example, from 1.0 mg to 100 mg of Compound 1, and from 25 mg to 800 mg of Compound 2. Again by way of example, compositions of the present invention can include from 30 mg to 50 mg of Compound 1, and from 100 mg to 400 mg of Compound 2. In a specific embodiment, some compositions of the present invention contain 40 mg of Compound 1, and from 75 mg to 800 mg of Compound 2. In another specific embodiment, some compositions of the present invention contain from 20 mg to 40 mg of Compound 1, and from 70 mg to 200 mg of Compound 2. In a further specific embodiment, some compositions of the present invention contain 40 mg of Compound 1, and 75 mg of Compound 2.

In another aspect, the present invention provides methods for the treatment of HCV infection in a human being, wherein each method includes the step of administering a therapeutically effective amount of a combination of Compound 1 and Compound 2 to a human being in need thereof, such as a human being infected with the hepatitis C virus. In the practice of the methods of this aspect of the invention, the combined amount of Compound 1 and Compound 2 is effective to treat HCV infection, although the amounts of Compound 1 and Compound 2 may also be individually effective to treat HCV infection. Compound 1 and Compound 2 may be administered together (e.g., in the form of a unit dosage, such as a tablet), or Compound 1 and Compound 2 may be administered separately. Compound 1 may be administered at the same time as Compound 2, or before or after the administration of Compound 2.

Typically, Compound 1 and Compound 2 are administered daily. In one embodiment, a daily dosage is administered in separate sub-doses, such as twice daily or three times per day. By way of example, in the practice of the methods of this aspect of the invention, an amount of from 1.0 mg to 100 mg of Compound 1, and from 25 mg to 800 mg of Compound 2 can be administered daily to a human being in need thereof. Again by way of example, an amount of from 30 mg to 50 mg (such as 40 mg) of Compound 1, and from 100 mg to 400 mg of Compound 2 can be administered daily to a human being in need thereof. Again by way of example, an amount of from 30 mg to 50 mg (such as 40 mg) of Compound 1, and from 70 mg to 400 mg of Compound 2 (such as 75 mg) can be administered daily to a human being in need thereof. The course of treatment can extend, for example, from 12 weeks to 48 weeks.

In one embodiment, Compound 1 and Compound 2 are administered orally (e.g., in the form of a tablet or capsule). In another embodiment, Compound 1 and Compound 2 are administered by injection, such as by intravenous injection. In another embodiment, Compound 1 and Compound 2 are administered by aerosol delivery.

Another aspect of the present invention includes the use of the combination of Compound 1 and Compound 2 in the manufacture of a medicament for the treatment of HCV infection in a human being.

Another aspect of the present invention includes a composition comprising Compound 1 and Compound 2 for use in the treatment or prevention of HCV infection in a human being.

The scope of the present invention includes all combinations of aspects and embodiments.

### DETAILED DESCRIPTION

Reference will now be made in detail to certain claims of the invention, examples of which are illustrated in the accompanying structures and formulas. While the invention will be described in conjunction with the enumerated claims, it will be understood that they are not intended to limit the invention to those claims. On the contrary, the invention is intended to cover all alternatives, modifications, and equivalents, which may be included within the scope of the present invention as defined by the claims.

All documents cited herein are each incorporated by reference in their entirety for all purposes.

When trade names are used herein, applicants intend to independently include the tradename product and the active pharmaceutical ingredient(s) of the tradename product.

As used herein, “Compound 1” means Compound 1 or a pharmaceutically acceptable salt, solvate, ester or stereoisomer thereof.

As used herein, “Compound 2” means Compound 2 or a pharmaceutically acceptable salt, solvate, ester or stereoisomer thereof.

As used herein, the term “therapeutically effective amount” refers to an amount of the combination of Compound 1 and Compound 2 that is effective to ameliorate at least one symptom of HCV infection in a human being. Thus, for example, in some HCV infected individuals a therapeutically effective amount of the combination of Compound 1 and Compound 2 is effective to reduce by a statistically significant amount the viral load of HCV viral particles present in the body of the infected person. Viral load can be measured, for example, by measuring plasma HCV RNA levels using, for example, the COBAS TaqMan HCV assay (Roche Molecular Systems). Typically, an HCV infected person who is treated with the combination of Compound 1 and Compound 2 in accordance with the present invention experiences an improvement in one or all of the symptoms associated with the HCV infection. For example, an HCV patient may experience an improvement in one or all of the following symptoms that can be associated with HCV infection: fever, headache, muscle aches, fatigue, loss of appetite, nausea, vomiting and diarrhea.

The present invention relates to methods, uses, and compositions comprising Compound 1 and. Compound 2. Compound 1 has the following structure:

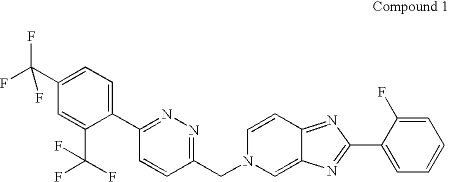

Compound 2 has the following structure:

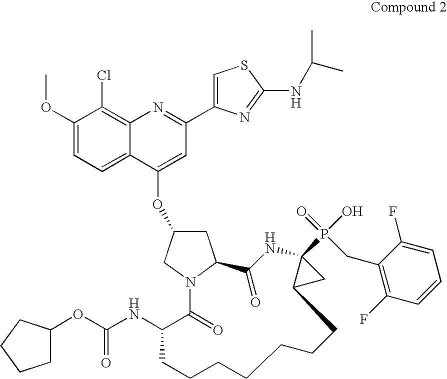

The data set forth in Example 3 show that Compound 1 and Compound 2 each have anti-HCV activity as measured in an in vitro HCV replicon assay, and that the combination of Compound 1 and Compound 2 has anti-HCV activity, as measured in the in vitro HCV replicon assay. Thus, both Compound 1 and Compound 2, and the combination of Compound 1 and Compound 2, are useful, for example, for inhibiting HCV replication in vitro and in vivo, such as inhibiting HCV replication in human beings infected with HCV. Compounds 1 and 2, and the combination thereof, can also be used, for example, in assays to identify additional molecules that inhibit, or otherwise affect, HCV replication in vitro or in vivo, or to study the mechanism of HCV replication in living cells.

Salt forms, or solvates, of Compounds 1 and 2 can be used in the practice of the present invention. Typically, but not necessarily, the salts of Compounds 1 and 2 are pharmaceutically acceptable salts. Salts encompassed within the term “pharmaceutically acceptable salts” refer to non-toxic salts of Compounds 1 and 2.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

The pharmaceutical compositions of the present invention include Compound 1 and Compound 2 in the pure state or in the form of a composition in which the compounds are combined with any other pharmaceutically compatible substance, which can be inert or physiologically active. The resulting pharmaceutical compositions can be used, for example, to treat HCV infection in a human being.

The manner in which Compounds 1 and 2 are administered can vary. For example, the compositions may be administered orally, such as in liquid form within a solvent such as an aqueous or non-aqueous liquid, or within a solid carrier. Compositions for oral administration include pills, tablets, capsules, caplets, syrups, and solutions, including hard gelatin capsules and time-release capsules. Standard excipients include binders, fillers, colorants, solubilizers and the like. Compositions can be formulated in unit dose form, or in multiple or subunit doses. Compositions including a liquid pharmaceutically inert carrier such as water or other pharmaceutically compatible liquids or semisolids can be used. The use of such liquids and semisolids is well known to those of skill in the art.

Again by way of example, the compositions can be administered via injection, i.e., intravenously, intramuscularly, subcutaneously, intraperitoneally, intraarterially, intrathecally; and intracerebroventricularly. Intravenous administration is the preferred method of injection. Suitable carriers for injection are well known to those of skill in the art and include, for example, 5% dextrose solutions, saline, and phosphate-buffered saline. The compounds can also be administered as an infusion or injection, namely, as a suspension or as an emulsion in a pharmaceutically acceptable liquid or mixture of liquids.

The compounds can also be administered directly to the respiratory tract by inhalation, namely, in the form of an aerosol either nasally or orally. Thus, one aspect of the present invention includes a novel, efficacious, safe, nonirritating, and physiologically compatible inhalable composition comprising Compound 1 and Compound 2 which are useful for treating HCV infection.

Other examples of delivery routes for Compounds 1 and 2 include rectal delivery, such as by the administration of a suppository, or transdermal administration.

Compounds 1 and 2 may be administered together or separately and, when administered separately, administration may occur simultaneously or sequentially, in any order. The amounts of Compounds 1 and 2, and the relative timings of administration, will be selected in order to achieve the desired therapeutic effect. The administration of Compound 1 and Compound 2 may be in combination by administration concomitantly in: (1) a unitary pharmaceutical composition including both compounds; or (2) separate pharmaceutical compositions each including one of the compounds. The combination may also be administered separately in a sequential manner wherein one treatment agent is administered first and the other second or vice versa. Such sequential administration may be close in time or remote in time.

Compounds 1 and 2 are typically administered in the form of pharmaceutical compositions that include at least one pharmaceutically acceptable carrier. The term “pharmaceutically acceptable carrier” as used herein means any material or substance formulated with the active ingredient in order to facilitate its preparation and/or its application or dissemination to the site to be treated. Suitable pharmaceutical carriers for use in the compositions of this invention are well known to those skilled in the art. They include additives such as wetting agents, dispersing agents, adhesives, emulsifying agents, solvents, glidants, coatings, antibacterial and antifungal agents (for example phenol, sorbic acid, chlorobutanol), and isotonic agents (such as sugars or sodium chloride), provided that the same are consistent with pharmaceutical practice, i.e. they are not toxic to mammals.

The pharmaceutical compositions of the present invention are prepared in any known manner, for instance by homogeneously mixing, coating and/or grinding the active ingredients in a one-step or multi-step procedure, with the selected carrier material and, where appropriate, other additives such as surface-active agents.

The pharmaceutical compositions of the present invention can include solubilized forms of Compound 1 and Compound 2, where Compounds 1 and 2 are dissolved in an appropriate solvent or solubilizing agent, or combinations thereof. The solvent typically includes various organic acids (typically C4-C24) such as capric, oleic or lauric acid. In addition, polyethylene glycols (PEGs) and/or short, medium, or long chain mono, di, or triglycerides can be employed to dissolve Compounds 1 and 2 for use in a liquid formulation. Pegylated short, medium or long chain fatty acids may also be used. Typically, the preparation is aqueous, i.e, water is the only solvent per se although it generally will also include the solubilizing agent such as an organic acid or the other agents described above.

The most common organic acids are the carboxylic acids whose acidity associated with the carboxyl group —COOH. Sulfonic acids, containing the group OSO₃H, are relatively stronger acids for use herein. In general, the acid desirably contains a lipophilic domain. Mono- or di-carboxylic acids are suitable.

Suitable surface-active agents optionally are used with any of the pharmaceutical compositions of this invention. Such agents also are known as emulgents or emulsifiers, and are useful in the pharmaceutical compositions of the present invention. They are non-ionic, cationic and/or anionic materials having suitable emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable from coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcoholamine salts of dodecylbenzene sulphonic acid or dibutyl-naphthalenesulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidyl-choline, dipalmitoylphoshatidyl-choline and their mixtures. Aqueous emulsions with such agents are within the scope of this invention.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, particularly halides, having 4 hydrocarbon radicals optionally substituted with halo, phenyl, substituted phenyl or hydroxy; for instance quaternary ammonium salts containing as N-substituent at least one C₈-C₂₂ alkyl radical (e.g. cetyl, lauryl, palmityl, myristyl and oleyl) and, as further substituents, unsubstituted or halogenated lower alkyl, benzyl and/or hydroxy-lower alkyl radicals.

A more detailed description of surface-active agents suitable for this purpose is found in “McCutcheon's Detergents and Emulsifiers Annual” (MC Publishing Crop., Ridgewood, N.J., 1981), “Tensid-Taschenbucw”, 2nd ed. (Hanser Verlag, Vienna, 1981) and “Encyclopaedia of Surfactants,” (Chemical Publishing Co., New York, 1981). Further details on techniques for formulation and administration may be found in the latest edition of Remington's Pharmaceutical Sciences (Maack Publishing Co, Easton, Pa.).

Compositions comprising Compound 1 and Compound 2 may be manufactured in a manner similar to that known in the art (e.g., by means of conventional mixing, dissolving, granulating, dragee-making, levigating, emulsifying, encapsulating, entrapping, or lyophilising processes). Compositions comprising Compound 1 and Compound 2 may also be modified to provide appropriate release characteristics, e.g., sustained release or targeted release, by conventional means (e.g., coating).

In another aspect, the present invention provides methods for the treatment of HCV infection in a human being, wherein each method includes the step of administering a therapeutically effective amount of a combination of Compound 1 and Compound 2 to a human being in need thereof, such as a human being infected with the hepatitis C virus. The pharmaceutical compositions of the present invention are useful in the practice of the treatment methods of the present invention.

In the practice of the methods of this aspect of the invention, the combined amount of Compound 1 and Compound 2 is effective to treat HCV infection, although the amounts of Compound 1 and Compound 2 may also be individually effective to treat HCV infection. Compound 1 and Compound 2 may be administered together (e.g., in the form of a unit dosage, such as a tablet), or Compound 1 and Compound 2 may be administered separately. Compound 1 may be administered at the same time as Compound 2, or before or after the administration of Compound 2.

Typically, Compound 1 and Compound 2 are administered daily. In one embodiment, a daily dosage is administered in separate sub-doses, such as twice daily or three times per day. By way of example, in the practice of the methods of this aspect of the invention, an amount of from 1.0 mg to 100 mg of Compound 1, and from 25 mg to 800 mg of Compound 2 can be administered daily to a human being in need thereof. Again by way of example, an amount of from 30 mg to 50 mg (such as 40 mg) of Compound 1, and from 100 mg to 400 mg of Compound 2 can be administered daily to a human being in need thereof. The course of treatment can extend, for example, from 12 weeks to 48 weeks, or such as, for example, from 12 weeks to 24 weeks.

The following examples merely illustrate the best mode now contemplated for practicing the invention, but should not be construed to limit the invention.

### Example 1a

### Synthesis of 5-({6-[2,4-bis(trifluoromethyl)phenyl]pyridazin-3-yl}methyl)-2-(2-fluorophenyl)-5H-imidazo[4,5-c]pyridine

Compound 1 has the IUPAC name: 5-({6-[2,4-bis(trifluoromethyl)phenyl]pyridazin-3-yl}methyl)-2-(2-fluorophenyl)-5H-imidazo[4,5-c]pyridine, and the CAS name: 5H-imidazo[4,5-c]pyridine, 5-[[6-[2,4-bis(trifluoromethyl)phenyl]pyridazin-3-yl]methyl]-2-(2-fluorophenyl).

In this method for making Compound 1, dimethoxyethane or its related solvents, all having the general formula R¹OR²O(R⁴O)ₐR³ wherein each of R¹, R², R³ and R⁴ are independently selected from C₁-C₆ alkyl and a is 0 or 1, have been found to be particularly advantageous over the conventional solvent DMF. Typically, each of R¹, R², R³ and R⁴ are independently C₁-C₂ alkyl and usually a is 0. C₁-C₆ alkyl includes fully saturated primary, secondary or tertiary hydrocarbon groups with 1 to 6 carbon atoms and thereby includes, but is not limited to methyl, ethyl, propyl, butyl, etc.

### Step 1

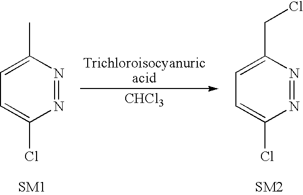

| Compound | MW | Amount | mmoles | Equivalents |
|---|---|---|---|---|
| SM1 | 128.56 | 5 g | 38.9 | 1 |
| TCCA | 232.41 | 3.62 g | 15.6 | 0.4 |
| CHCl₃ | | 130 mL | | |

To a solution of the commercially available starting material (SM1) in CHCl₃, trichloroisocyanuric acid (TCCA) was added at 60° C. Then the solution was stirred for 1.5 hrs., cooled down and filtered with HiFlo-Celite. The filtrate was concentrated and dried with vacuum. The yield was 5.037 g of SM2.

### Step 2

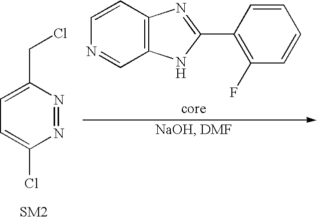

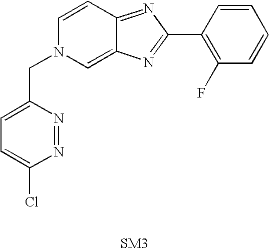

| Compound | MW | Amount | mmoles | Equivalents |
|---|---|---|---|---|
| SM2 | 163 | 5.073 g | 31.12 | 1 |
| Core | 213.2 | 6.635 g | 31.12 | 1 |
| NaOH (10%) | 40 | 1.245 g | 31.12 | 1 |
| DMF | | 320 mL | | |

To a solution of the starting material designated as “core” (obtained as described below) in DMF (dimethylformamide), NaOH was added. Then SM2 (obtained from step 1) was dissolved in DMF (20 mL) and added to the solution slowly. The reaction was stirred for 3 hrs, was diluted with water and extracted with EtOAc. The organic layer was dried with Na₂SO₄. The solvent was removed and the product recrystallized with DCM (dichloromethane). The yield was 5.7 g of SM3.

### Step 3

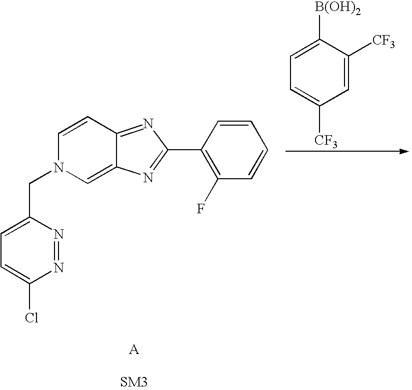

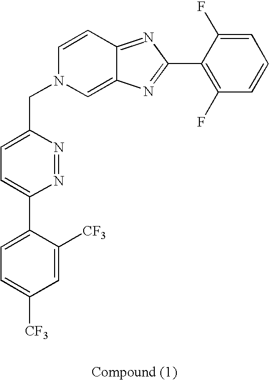

| Compound | MW | Amount | Moles | Equivalents |
|---|---|---|---|---|
| SM3 | 453.79 | 95 mg | 0.209 | 1 |
| DME | 500 μL | | | |
| 2N aq. Na₂CO₃ | | 313 μL | 0.626 | 3 |
| 2,4-bisCF₃- | 257.93 | 80.9 mg | 0.313 | 1.5 |
| phenylboronic acid | | | | |
| Pd(PPh₃)₄ | 1155 | 12 mg | 0.0104 | 0.05 |

The compound designated as “SM3” was dissolved in dimethoxyethane (DME). To this solution was added 2,4-bis(trifluromethyl)phenylboronic acid and a 2N aq. Na₂CO₃ solution. To the resulting biphasic mixture was added Pd(PPh₃)₄ and the reaction was then heated at 80° C. for 72 hrs. The reaction was cooled to room temperature and filtered through Celite and the Celite washed with EtOAc. The filtrate was concentrated in vacuo. The residue was purified on 6 g SiO2 using MeOH/CH2Cl2 to elute compound. The compound thus obtained was contaminated with PPh₃(O). The product was repurified on a 1 mm Chromatotron plate with 0 to 5% MeOH/CH₂Cl₂ in 1% steps. The pure fractions were combined and concentrated in vacua, then dried on high vacuum for 12 hrs. 11.8 mg of the free base of compound (1) was obtained with no PPh₃ contamination.
¹H NMR (300 MHz,CD₃OD)
6.20 (s, 2)
7.32 (m, 3)
7.52 (m, 1)
7.78 (d, 1)
7.89 (d, 1)
7.95 (s, 2)
8.15 (m, 3)
8.35 (d, 1)
9.12 (s, 1)
LC/MS M+H=518

### Example 1b

### Synthesis of 5-({6-[2,4-bis(trifluoromethyl)phenyl]pyridazin-3-yl}methyl)-2-(2-fluorophenyl)-5H-imidazo[4,5-c]pyridine

This example is directed to an additional method for making compound (1), employing the following schemes.

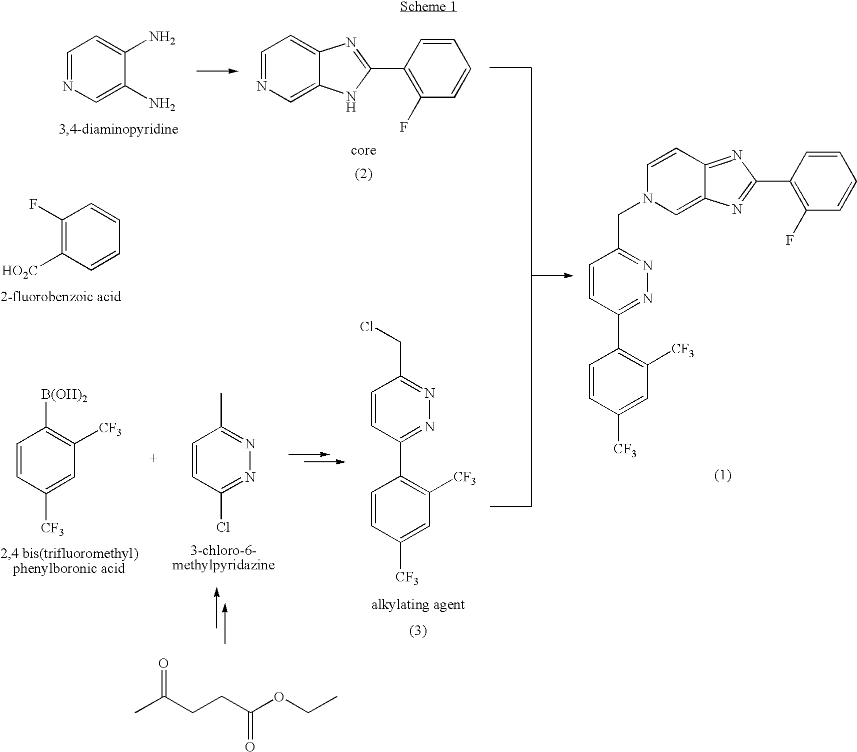

### Process Summary

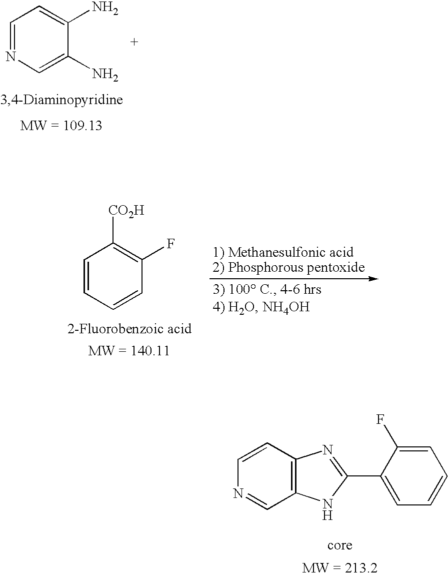

Methanesulfonic acid was added to 2-fluorobenzoic acid in a reactor with active cooling keeping T≦50° C. 3,4-Diaminopyridine was then added portionwise to this cooled slurry, keeping T≦35° C. The contents of the reactor were then heated to 50° C. Phosphorus pentoxide was added in a single charge. The reaction was then heated at 90-110° C. for at least 3 hours. The reaction was sampled for completion by HPLC analysis. The reaction was cooled to ambient temperature and water was added portionwise slowly to quench the reaction. The reaction was then diluted with water. In solubles were removed by filtration. The pH of the filtrate was adjusted to 5.5-5.8 with ammonium hydroxide. The reaction was allowed to self-seed and granulate for ˜4 hours at ambient temperature. The pH was then adjusted to 8.0-9.3 with ammonium hydroxide. The slurry was held at ambient temperature for at least 2 hours. The solids were isolated by filtration and washed with water, followed by IPE. The wet cake was dried in vacuo at not more than 60° C. until ≦1% water remains. The dry product is the compound designated as “core”.

| Summary of Materials | M.W. | Wt. Ratio | Mole ratio |
|---|---|---|---|
| 3,4-Diaminopyridine | 109.13 | 1.0 | 1.0 |
| 2-Fluorobenzoic acid | 140.11 | 1.4 | 1.1 |
| Methanesulfonic acid | 96.1 | 7.0 | 8.0 |
| Phosphorus pentoxide | 141.94 | 1.3 | 1.0 |
| Water | 18.02 | 40 | — |
| Isopropyl ether | 102.17 | 5.0 | — |
| Ammonium hydroxide | 35.09 | ~10 | — |

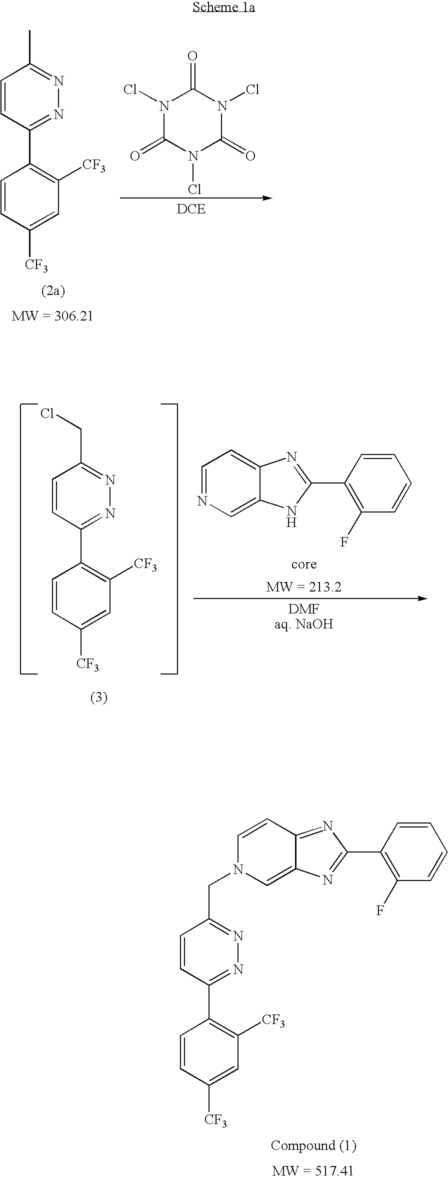

A solution of 2a in 1,2-dichloroethane was heated to 40-45° C. Trichloroisocyanuric acid was added and the mixture was heated at 60-70° C. for at least 2 hours. The reaction was sampled for completion by HPLC analysis. The reaction was cooled to ambient temperature. Celite was added to absorb insolubles, then solids were removed by filtration. The filtrate was washed with 0.5 N sodium hydroxide solution. The organic layer was concentrated to lowest stirrable volume and displaced with DMF. The compound designated as “core” and 10% aqueous sodium hydroxide solution were added. The reaction was stirred at ambient temperature for at least 8 hours. The reaction was sampled for completion by HPLC analysis. An additional 10% charge of 10% sodium hydroxide solution was added to the reaction. The reaction was then charged into water to isolate the crude product, compound (1). After granulating for at least 1 hour, the solids were isolated and washed with water and isopropyl ether. The wet cake was recrystallized from ethyl acetate to afford low melt (˜220° C.) Compound (1) (polymorph I). The wet-cake was then reslurried in ethyl acetate in the presence of less than about 0.5% water to obtain the high melt (˜236° C.) Compound (1) (polymorph II). The solids were collected by filtration and washed with ethyl acetate. The wet cake was dried in vacuo at not more than 60° C. to obtain the dry crystalline polymorph II.

| | | Wt. | |
|---|---|---|---|
| Summary of Materials | M.W. | Ratio | Mole ratio |
| 3-chloro-6-methylpyridazine | 128.56 | 1.0 | 1.0 |
| 2,4bis(trifluromethyl)phenylboronic acid | 257.93 | 4.0 | 2.0 |
| X-Phos | 476.72 | 0.18 | 0.05 |
| Palladium acetate | 224.49 | 0.04 | 0.025 |
| 1,2-Dimethoxyethane | 90.12 | 16.7 | — |
| Potassium carbonate | 138.21 | 2.15 | 2.0 |
| Water | 18.02 | 7.8 | — |
| Copper iodide | 190.45 | 0.037 | 0.025 |
| Celite | — | 0.25 | — |
| Heptane | 100.2 | 22.4 | — |

### Example 2

### Preparation of Compound 2

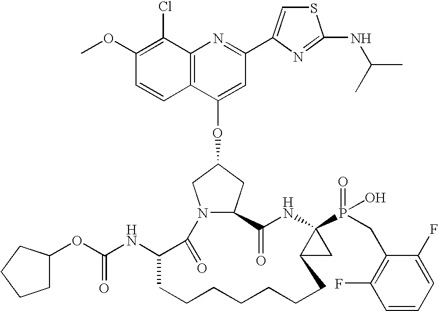

Step 1. Intermediate XI has the following structure:

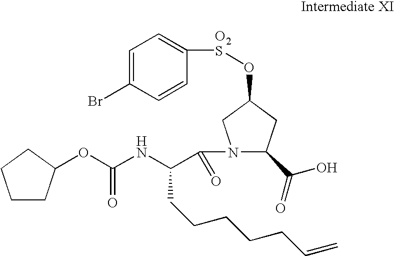

Intermediate XI (17.42 g, 28.30 mmol) was dissolved in THF (136 mL) and cooled to 0° C. To the solution was added N-methylmorpholine (4.7 mL, 42.7 mmol). After 10 min at 0° C., i-butylchloroformate (4.05 mL, 30.96 mmol) was added dropwise. After an additional 1 h, (1-amino-2-vinyl-cyclopropyl)-(2,6-difluoro-benzyl)-phosphinic acid ethyl ester (8.94 g, 29.70 mmol) was slowly added as a soln in THF (20 mL). The structure of (1-amino-2-vinyl-cyclopropyl)-(2,6-difluoro-benzyl)-phosphinic acid ethyl ester is shown below as Intermediate XII.

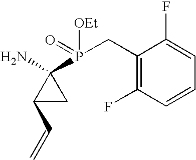

The suspension was warmed to room temperature and after 2 h it was partitioned between H₂O (400 mL) and ethylacetate (200 mL). The aqueous layer was extracted with ethylacetate (200 mL×2) and the combined organic layers were washed with HCl (1N, 225 mL) and H₂O (200 mL). The acid wash and aqueous wash were combined and back-extracted with ethylacetate (175 mL×2, 100 mL×2). The combined organic layers were washed with brine (400 mL), dried over Na₂SO₄, and concentrated in vacuo providing 25.06 g of diene product in 98.5% crude yield. LCMS (M+1): 898.06

Step 2. The crude diene product (12.91 g, 14.36 mmol) was dissolved in CH₂Cl₂ (1440 mL) and the solution was degassed for 30 min. The solution was heated to 40° C. and Grubb's G1 catalyst (2.95 g, 3.59 mmol) was added. The reaction was refluxed for 17 h whereupon tris-hydroxymethylphosphine (22.3 g, 18.0 mmol), TEA (50 mL, 35.9 mmol), and H₂O (400 mL) were added and the reaction mixture was heated to reflux for an additional 16 h. The reaction mixture was cooled to room temperature and the two layers were separated. The organic layer was washed with H₂O (400 mL) and brine (300 mL), dried over MgSO₄, and concentrated. The crude residue was purified by silica-gel chromatography to afford 8.30 g of macrocyclic olefin product in 66% yield. LCMS (M+1): 870.09.

Step 3. The macrocyclic olefin (7.34 g, 8.42 mmol) was dissolved in ethylacetate (105 mL) and rhodium on alumina (5% wt, 2.945 g, 0.40 wt %) was added. The system was evacuated and flushed with H₂ (1 atm, 3×). To the system, after 3 h, was added more rhodium on alumina (5% wt, 842 mg, 0.10 wt %) and it evacuated and flushed with H₂ (1 atm, 3×). After an additional 1 h the suspension was filtered and concentrated in vacuo providing 6.49 g of reduced macrocycle in 88% crude yield. LCMS (M+1): 872.04.

Step 4. The brosylate macrocycle (6.49 g, 7.67 mmol) was dissolved in N-methylpyrrolidinone (25.0 mL) and 8-chloro-2-(2-isopropylamino-thiazol-4-yl)-7-methoxy-quinolin-4-ol (2.564 g, 7.33 mmol) followed by Cs₂CO₃ (4.40 g, 13.50 mmol) were added. The mixture was heated to 65° C. for 6 h then diluted with ethylacetate (200 mL) and washed with LiCl (5%, 250 mL). The aqueous layer was extracted with ethylacetate (100 mL×2) and the combined organic layers were washed with brine (150 mL), dried over Na₂SO₄/MgSO₄, and concentrated in vacuo. The crude residue was purified via silica-gel chromatography (ethylaceate-methanol) affording 4.39 g of aminothiazole product in 58% yield. LCMS (M+1): 985.28.

Step 5. Phosphinate ester (23.7 g, 24.05 mmol) was dissolved in CH₃CN (240 mL) and cooled to 0° C. Iodotrimethylsilane (17.4 mL, 122.3 mmol) was added at a fast drop-wise pace followed by, after 10 min, 2,6-lutidine (17.0 mL, 146.4 mmol). The reaction mixture was slowly warmed to room temperature and stirred for 1 h then cooled back down to 0° C. and 2,6-Lutidine (11.1 mL, 95.6 mmol) followed by MeOH (24 mL) were added. The solution was concentrated in vacuo and the crude residue was purified by HPLC to afford 12.68 g of Compound 2 in 55% yield. ¹H NMR (300 MHz, CDCl₃) δ 8.35 (d, J=9.3 Hz, 1H), 8.28 (s, 1H), 7.85 (s, 1H), 7.64 (d, J=9.6 Hz, 1H), 7.35-7.22 (m, 1H), 7.02-6.89 (m, 2H), 5.85 (bs, 1H), 4.82-4.71 (m, 2H), 4.33 (bs, 1H), 4.28-3.99 (m, 3H), 4.16 (s, 3H), 3.57-3.28 (m, 2H), 2.90-2.78m, 1H), 2.63-2.50 (m, 1H), 2.08-1.91 (m, 1H), 1.91-170 (m, 2H), 1.70-1.13 (m, 22H), 1.37 (d, J=6.9 Hz, 6H); ³¹P NMR (121.4 MHz, CD₃OD) δ 42.4; LCMS (M+1): 957.35. g.

### Example 3

### Anti-HCV Activity of the Combination of Compound 1 and Compound 2

### Materials and Methods.

Compound 1 and Compound 2 were synthesized by Gilead Sciences (Foster City, Calif.).

HCV genotype 1b replicon cells (Huh-luc) were obtained from Reblikon (Mainz, Germany). The replicon in these cells is designated I389luc-ubi-neo/NS3-3′/ET and encodes a selectable resistance marker (neomycin phosphotransferase) as well as the firefly luciferase reporter gene. Huh-luc cells were maintained in Dulbecco's Modified Eagle's Medium (DMEM; GIBCO, Carlsbad, Calif.) supplemented with 10% fetal bovine serum (FBS; Hyclone, Logan, Utah) and 0.5 mg/mL of G-418 (GIBCO). Cells were passaged twice a week and maintained at subconfluent levels.

Replicon cells were seeded in 96-well plates at a density of 5×10³ cells per well in 100 μL of DMEM culture medium, excluding G-418. Compounds 1 and 2 were serially diluted 1:3 in 100% DMSO (Sigma). These serial dilutions were added to the cells at a 1:200 dilution to achieve a final concentration of 0.5% DMSO in a total volume of 200 μL. Plates were incubated at 37° C. for 3 days, after which culture media were removed and cells were lysed and assayed for luciferase activity using a commercial luciferase assay (Promega, Madison, Wis.). HCV replication levels in drug-treated samples were expressed as a percentage of those in untreated controls (defined as 100%), and data were fit to the logistic dose response equation y=a/(1+(x/b)c) using XLFit4 software (IDBS, Emeryville, Calif.). EC50 values were calculated from the resulting equations as described previously (Delaney, W. E., et al., Antimicrobial Agents Chemotherapy, 45(6):1705-1713 (2001)).

Replicon cells were seeded in 96-well plates at a density of 5×10³ cells per well in 100 μL of culture medium. Compounds 1 and 2 were serially diluted in 100% DMSO as described above and added in a matrix format to 96-well plates, achieving a defined set of different drug concentrations and ratios in a final volume of 200 μL and a final DMSO concentration of 0.5%. For each individual drug, the EC50 value was selected as the midpoint for the concentration range tested. Cells were incubated for three days and analyzed for luciferase expression as indicated above. For the combination study, two independent experiments were performed in triplicate.

Data were analyzed using the MacSynergy II program developed by Prichard and Shipman (Prichard M N, Aseltine K R, Shipman C, Jr., MacSynergy™ II, Version 1.0. University of Michigan, Ann Arbor, Mich., 1993; Prichard M. N., Shipman C., Jr., Antiviral Res 14 (4-5):181-205 (1990); Prichard M. N., Shipman C, Jr., Antivir Ther 1 (1):9-20 (1996); Prichard M. N., et al., Antimicrob Agents Chemother 37 (3):540-5 (1993). The software calculates theoretical inhibition assuming an additive interaction between drugs (based on the Bliss Independence model) and quantifies statistically significant differences between the theoretical and observed inhibition values. Plotting these differences in three dimensions results in a surface where elevations in the Z-plane represent antiviral synergy and depressions represent antiviral antagonism between compounds. The calculated volumes of surface deviations are expressed in nM2%. Per Prichard and Shipman, combination effects are defined as:

Highly synergistic if volumes >100 nM².

Slightly synergistic if volumes are >50 and ≦100 nM².

Additive if volumes are >−50 nM² and ≦50 nM².

Slightly antagonistic if volumes are >−100 nM² and ≦−50 nM².

Antagonistic if volumes are ≦−100 nM².

### Results.

Prior to initiating combination experiments, EC₅₀ values in Huh-luc replicon cells were determined for Compound 1 and Compound 2 (Table 1). Both compounds had an antiviral effect.

TABLE 1
Individual EC50s for Anti-HCV Compounds 1 and 2 in Huh-luc
Replicon Cells

| Compound | EC₅₀ (nM)^{a} |
|---|---|
| Compound 1 | 3 ± 2 |
| Compound 2 | 11 ± 3 |

^{a}EC₅₀ indicates average ± standard deviation for two or more independent experiments.

The antiviral effect of the combination of Compound 1 and Compound 2 was measured, and the resulting data were analyzed using MacSynergy II, which provides surface plots displaying significant deviations from additivity. Quantification of statistically significant deviations from additivity indicated that the combination of Compounds 1 and 2 had synergy/antagonism volumes between −50 nM² and 50 nM² indicating additive antiviral effects (Table 2).

TABLE 2
Quantification of Antiviral Synergy and Antagonism and Drug
Interactions for Combination of Compound 1 and Compound 2

| Drug(s) Used in | | | |
|---|---|---|---|
| Combination with | Synergy | Antagonism | |
| Compound 2 | Volume (nM²)^{a} | Volume (nM²)^{a} | Interaction |
| Compound 1 | 13.5 ± 10.5 | 0.07 ± 0.07 | Additive |

^{a}Values represent the mean ± standard deviation of two independent experiments performed in triplicate

The results of the in vitro experiments set forth in Table 2 indicate that Compound 2 has additive antiviral activity when combined with Compound 1.

### Example 4

### Cross Resistance between Compound 1 and Compound 2

The results described in this Example demonstrate the cross-resistance profiles of Compound 1 and Compound 2.

### Materials and Methods.

Compound 1 and Compound 2 were synthesized at Gilead Sciences, Inc. (Foster City, Calif.). Dulbecco's Modified Eagle Medium (DMEM) (#10569-010) and Geneticin (G418) (#10131-035) were purchased from GIBCO (Carlsbad, Calif.). Fetal bovine serum (FBS) (#ASL30941) was purchased from Hyclone (Logan, Utah). The luciferase assay system (#E1501) was purchased from Promega (Madison, Wis.).

HCV genotype 1b-PI-luc, a bi-cistronic replicon and cured Huh7 cells (Lunet) were obtained from Ralf Bartenschlager, University of Heidelberg (Lohmann V, et al., Science 285 (5424):110-3 (1999)). The 1b-PI-luc construct contains a luciferase reporter gene driven by the poliovirus IRES and the HCV nonstructural genes from 1b-Con1 driven by the EMCV IRES. Three adaptive mutations, T1280I and E1202G in NS3 and K1846T in NS4B, were introduced into this construct for efficient replication. Cured Huh-7 cells are designated Huh-lunet and were obtained by curing HCV replicon-containing Huh-7 cells with IFNα. Cells were maintained in DMEM medium supplemented with 10% FBS and passaged twice a week before reaching confluent levels.

Mutations were introduced into the HCV replicon plasmids using Stratagene's QuikChange II XL mutagenesis kit, following the manufacturer's instructions. Mutations were confirmed by di-deoxy sequencing using appropriate primer pairs.

Replicon RNA was transfected into Lunet cells following the protocol provided by R. Bartenschlager (Lohmann V, et al., Science 285 (5424):110-3 (1999)). Briefly, cells were trypsinized and washed twice with PBS. A suspension of 4×10⁶ cells in 400 μL of PBS was mixed with 5-10 μg of RNA and subjected to electroporation using settings of 960 μF and 270V. Cells were quickly transferred into 20 mL of pre-warmed culture medium and seeded into 96-well plates at appropriate densities. Cell suspensions were seeded in a 96-well plate at 100 μL/well and allowed to attach overnight. An aliquot of each cell suspension was taken 4 hours post-transfection and luciferase activity was measured to normalize the transfection efficiencies. For EC50 determinations, compounds were serially diluted in 100% DMSO and then added to cells at 1:200 dilutions, achieving a final concentrations of 0.5% DMSO and a total volume of 200 μL. Cells were cultured for three days at 37° C., after which the culture medium was removed and the Firefly luciferase activity was measured using the Luciferase assay system (Promega, Madison, Wis.) with a Victor Luminometer (PerkinElmer, Waltham, Mass.).

EC₅₀ data were analyzed using the GraphPad Prism software package (GraphPad Software, La Jolla, Calif.). EC₅₀ values were calculated using nonlinear regression analysis.

### Results.

To determine the susceptibilities of the major HCV NS5B polymerase inhibitor resistance mutations to Compound 2, transient transfection assays were performed using wild-type and mutant 1b Con-1 PI-Luc replicons. As shown in Table 3, expected levels of resistance were observed for Compound 1 for this panel of mutant replicons. Compound 2 retained wild-type activity against all mutations (≦2.1-fold change in EC₅₀ values from wild-type), indicating no cross-resistance of Compound 2 with Compound 1.

TABLE 3
Activity of Compound 2 Against Known HCV NS5B Polymerase
Mutations

| | | Fold Resistance* | | | |
|---|---|---|---|---|---|
| | EC₅₀ (nM) | | | | C316Y/C445F/ |
| Compound | WT | M423T | M414T | Y448H | Y452H |
| Compound 2 | 50.7 | 1.1 | 2.0 | 1.0 | 2.1 |
| Compound 1 | 1.5 | 0.8 | 0.7 | 38.0 | >343 |

*Data was derived from at least two independent experiments per mutant, and testing was performed in duplicate within each experiment. Fold resistance was calculated as the ratio of mutant EC₅₀ to wild type EC₅₀.
WT = wild type.

The susceptibility of a panel of protease inhibitor resistance-associated-mutations to Compound 1 was determined using a transient replication assay. As shown in Table 4, the expected levels of resistance were observed with each of these mutants for the HCV NS3 protease inhibitor Compound 2. In contrast, all mutants remained sensitive to Compound 1 (≦1.2-fold change in EC₅₀ values from wild-type), consistent with a mechanism of action of Compound 1 that is distinct from HCV NS3 protease inhibitors.

TABLE 4
Activity of Compound 1 against NS3 Protease Mutations

| | EC₅₀ (nM) | Fold Resistance* | | | | | |
|---|---|---|---|---|---|---|---|
| Compounds | WT | V36M | T54A | R155K | A156T | D168E | D168V |
| Compound 1 | 0.79 | 1.1 | 1.0 | 0.9 | 0.6 | 1.0 | 1.2 |
| Compound 2 | 21.37 | 3.0 | 0.6 | 566.3 | 1882.4 | 120.0 | 1832.3 |

*Data was derived from two independent experiments per mutant, duplicate in each experiment. Resistance fold is calculated as the ratio of mutant EC₅₀ to wild type EC₅₀;
WT = wild type.

Compound 2 is a potent inhibitor of HCV NS3/4A protease, while Compound 1 is a novel non-nucleoside polymerase inhibitor. The data disclosed in this Example show that Compound 2 displayed wild-type potency against a panel of known mutants in NS5B that confer resistance to Compound 1. Conversely, Compound 2 showed wild-type susceptibility to Compound 1.

### Example 5

### Enhancement of in vivo Exposure to Compound 1 by Compound 2

The results described in this Example demonstrate that the exposure to Compound 1 in human beings is increased by coadministration of Compound 2.

The study was designed to evaluate potential interactions between Compound 1 and Compound 2 following multiple-dose administration in healthy subjects who were assigned to one of two groups (either Group 1 or Group 2).

### Group 1 (N=15):

Administration of 40 mg Compound 1 every morning and evening, approximately 12 hours apart, for 7 days (Days 1 to 7), followed by coadministration of 40 mg Compound 1 and 75 mg Compound 2 every morning and evening, approximately 12 hours apart, for 6½ days (Days 8 to 14 (am)).

Serial blood samples for analysis of plasma concentrations of Compound 1 were collected up to 12 hours after the morning dose on Day 7, and up to 120 hours after the dose on Day 14 to determine systemic drug exposure.

### Group 2 (N=10):

Administration of 75 mg Compound 2 every morning and evening, approximately 12 hours apart, for 13 doses (last dose was day 7 in AM), followed by a washout interval of 7 days (Days 8 to 14), then coadministration of 75 mg Compound 2 and 40 mg Compound 1 every morning and evening, approximately 12 hours apart, for 13 doses (last dose was day 21 in AM).

Serial blood samples for analysis of plasma concentrations of Compound 2 were collected up to 120 hours after the morning dose on Days 7 and 21 to determine systemic drug exposure.

Results for Group 1 are summarized in Table 5, and results for Group 2 are summarized in Table 6.

TABLE 5
Mean (CV %) Compound 1 Pharmacokinetic Parameters
Following Oral Administration of Compound 1 Alone and in
Combination with Compound 2

| | | | % Geometric Least |
|---|---|---|---|
| | Mean^{a} (% CV) | | Square |
| Compound | | Compound 1 (40 mg | Mean Ratio^{b} (90% CI) |
| 1 PK | Compound 1 | BID) + | Compound 1 + |
| Parameter | (40 mg BID) | Compound 2 (75 mg | Compound 2/ |
| (N = 14) | Alone | BID) | Compound 1 alone |
| Cₘₐₓ | 5.04 (43) | 9.47 (45) | 185.2 (167.3, 205.0) |
| (μg/mL) | | | |
| Cₜₐᵤ | 2.57 (44) | 6.61 (52) | 251.2 (219.5, 287.5) |
| (μg/mL) | | | |
| AUCₜₐᵤ | 43.39 (44) | 89.58 (44) | 206.4 (185.1, 230.0) |
| (μg · hr/mL) | | | |

CV = coefficient of variation,
BID = twice daily,
CI = confidence interval
^{a}Arithmetic mean
^{b}Geometric least squares means are obtained by the back-transformation of least-squares means of the parameters from an ANOVA using a mixed model based on the natural logarithmic scale. Geometric least squares mean ratio of 100% indicates no change in the parameters.

Based on Geometric least squares mean ratio, coadministration of Compound 2 (75 mg BID) in combination with Compound 1 (40 mg BID) resulted in an 85% increase in maximal observed concentration (Cₘₐₓ), a 151% increase in observed concentration at the end of the dosing (Cₜₐᵤ), and 106% increase in average overall systemic exposure (AUCₜₐᵤ) for Compound 1 compared to administration of Compound 1 at 40 mg BID alone. AUCₜₐᵤ is used as the measure for overall systemic exposure in multiple dosing situations, and AUC_{inf} is used in single dose situations.

TABLE 6
Mean (CV %) Compound 2 Pharmacokinetic Parameters
Following Oral Administration of Compound 2 Alone and in
Combination with Compound 1

| | | | % Geometric Least |
|---|---|---|---|
| | Mean^{a} (% CV) | | Square |
| Compound | | Compound 2 (75 mg | Mean Ratio^{b} (90% CI) |
| 2 PK | Compound 2 | BID) + | Compound 1 + |
| Parameter | (75 mg BID) | Compound 1 (40 mg | Compound 2/ |
| (N = 9) | Alone | BID) | Compound 2 alone |
| Cₘₐₓ | 4.50 (87) | 3.43 (86) | 80.0 (66.7, 95.6) |
| (μg/mL) | | | |
| Cₜₐᵤ | 1.55 (110) | 0.95 (107) | 70.0 (55.9, 87.5) |
| (μg/mL) | | | |
| AUCₜₐᵤ | 35.30 (93) | 25.10 (91) | 75.9 (62.0, 93.0) |
| (μg · hr/mL) | | | |

CV = coefficient of variation,
BID = twice daily,
CI = confidence interval
^{a}Arithmetic mean
^{b}Geometric least squares means are obtained by the back-transformation of least-squares means of the parameters from an ANOVA using a mixed model based on the natural logarithmic scale. Geometric least squares mean ratio of 100% indicates no change in the parameters.

Based on Geometric least squares mean ratio, coadministration of Compound 1 (40 mg BID) in combination with Compound 2 (75 mg BID) resulted, in a 20% decrease in maximal observed concentration (Cₘₐₓ), a 30% decrease in observed concentration at the end of the dosing (Cₜₐᵤ), and 24% decrease in average overall systemic exposure (AUCₜₐᵤ) for Compound 2 compared to administration of Compound 2 (75 mg BID) alone.

These results show that steady-state plasma exposure of Compound 1, following administration of Compound 1 at 40 mg BID, was increased significantly for all measure of drug exposure (i.e., Cₘₐₓ, AUCₜₐᵤ, and Cₜₐᵤ), when coadministered with Compound 2 at 75 mg BID. These results demonstrate the ability of Compound 2 to boost drug exposure of Compound 1.

Glossary of Abbreviations and Definitions of Terms Used in Example 5
AUCₜₐᵤ The area under the concentration versus time curve over the dosing interval
AUC₀₋ₗₐₛₜ Area under the concentration versus time curve from time zero to the last quantifiable concentration
AUC_{inf} Area under the concentration versus time curve extrapolated to infinite time, calculated as AUC₀₋ₗₐₛₜ+(Cₗₐₛₜ/λ_{z})
BID twice daily
CI confidence interval
Cₘₐₓ The maximum observed concentration of drug in plasma
Cₜₐᵤ The observed drug concentration at the end of the dosing interval
CV coefficient of variation

### Example 6

An additional pharmacokinetic drug-drug interaction study was conducted to further evaluate the pharmacokinetic interaction potential between Compound 1 and Compound 2 at a higher Compound 2 dose.

### Study Design:

This study was designed to characterize both the effect of multiple-dose administration of Compound 2 on the single dose pharmacokinetics of Compound 1 (at doses of 20 mg and 40 mg) and the effect of multiple-dose administration of Compound 1 on the single dose pharmacokinetics of Compound 2. In this study, healthy subjects were randomized into one of the following three treatment groups:

Treatment Groups 1 and 2 (N=15/group):
Group 1: single dose of Compound 1 at 20 mg (Day 1), Compound 2 at 150 mg BID for 3 days (Days 8-10), Compound 2 at 150 mg+Compound 1 at 20 mg (single morning dose) followed by Compound 2 at 150 mg alone in the evening (Day 11), and then Compound 2 at 150 mg BID alone for 3 days (Days 12-14)
Group 2: identical to Group 1 except that all Compound 1 doses were 40 mg

Serial blood samples for analysis of plasma concentrations of Compound 1 were collected up to 96 hours post-morning dose on Days 1 and 11 to determine systemic drug exposure.

### Treatment Group 3 (N=20):

Group 3: single dose of Compound 2 at 75 mg (Day 1), Compound 1 at 40 mg BID for 6 days (Days 4-9), Compound 1 at 40 mg+Compound 2 at 75 mg (single morning dose) followed by Compound 1 at 40 mg alone in the evening (Day 10), and then Compound 1 at 40 mg BID alone for 2 days (Days 11-12)

Serial blood samples for analysis of plasma concentrations of Compound 2 were collected up to 72 hours post-morning dose on Days 1 and 10 to determine systemic drug exposure.

### Study Results for Groups 1 and 2:

Single-dose plasma pharmacokinetic parameters of Compound 1 after administration of a single dose of Compound 1 (20 or 40 mg) alone and with multiple doses of Compound 2 (150 mg BID) are presented in Table 7.

TABLE 7
Compound 1 Pharmacokinetic Parameters and Statistical
Comparisons of the Parameters, Groups 1 and 2, Preliminary Data

| | | | % Geometric Least- |
|---|---|---|---|
| | | | Squares Mean |
| | | | Ratio^{c} (90% CI) |
| | Mean^{b} (% CV) | | Compound 1 + |
| Compound 1 PK | | Compound 1 + | Compound 2/ |
| Parameter | Compound 1 Alone | Compound 2 | Compound 1 alone |

Group 1: single-dose Compound 1 at 20 mg and multiple-dose Compound 2 at 150 mg
BID (N = 15)

| | | | |
|---|---|---|---|
| Cₘₐₓ (μg/mL) | 0.94 (25) | 1.14 (18) | 123.1 (111.6, 135.7) |
| AUC₀₋ₗₐₛₜ (μg * hr/mL) | 12.85 (44) | 32.61 (42) | 250.5 (217.8, 288.0) |
| AUC_{inf} (μg * hr/mL) | 13.39 (38) | 49.53 (68) | 321.1 (262.8, 392.2) |
| T_{1/2} (hr)^{a} | 16.61 (9.61, 28.07) | 35.81 (12.76, 156.99) | N/A |

Group 2: single-dose Compound 1 at 40 mg and multiple-dose Compound 2 at 150 mg
BID (N = 15)

| | | | |
|---|---|---|---|
| Cₘₐₓ (μg/mL) | 1.98 (29) | 2.73 (35) | 136.8 (122.7, 152.6) |
| AUC₀₋ₗₐₛₜ (μg * hr/mL) | 34.11 (34) | 87.53 (37) | 253.0 (229.3, 279.1) |
| AUC_{inf} (μg * hr/mL) | 35.47 (34) | 136.88 (55) | 350.4 (300.2, 409.1) |
| T_{1/2} (hr)^{a} | 19.21 (11.43, 30.84) | 39.29 (23.27, 113.54) | N/A |

CV = coefficient of variation,
BID = twice daily,
CI = confidence interval,
N/A = not applicable
^{a}Median (min, max)
^{b}Arithmetic mean
^{c}Geometric least squares means are obtained by the back-transformation of least-squares means of the parameters from an ANOVA using a mixed model based on the natural logarithmic scale. Geometric least squares mean ratio of 100% indicates no change in the parameters.

Based on geometric least-squares mean ratio, for Group 1, maximal observed concentration (Cₘₐₓ), average systemic exposure to the last measurable concentration (AUC₀₋ₗₐₛₜ), and average overall systemic exposure (AUC_{inf}) of Compound 1 showed an increase of 23%, 151%, and 221%, respectively, when 20 mg of Compound 1 was coadministered with multiple doses of Compound 2 (150 mg BID) compared with administration of single-dose Compound 1 at 20 mg alone. Similar magnitudes of increase in Compound 1 pharmacokinetic parameters were observed from Group 2 where Compound 1 was administered as a single dose of 40 mg (37%, 153% and 250% increase in Cₘₐₓ, AUC₀₋ₗₐₛₜ, and AUC_{inf} of Compound 1, respectively). In addition, an approximately 2-fold increase in median Compound 1 terminal phase elimination half-life was observed for both groups when given with Compound 2 at 150 mg BID compared with administration of Compound 1 alone.

### Study Results for Group 3:

Single-dose plasma pharmacokinetic parameters of Compound 2 after administration of a single dose of Compound 2 at 75 mg alone and with multiple doses of Compound 1 (40 mg BID) are presented in Table 8.

TABLE 8
Compound 2 Pharmacokinetic Parameters and Statistical
Comparisons of the Parameters, Group 3, Preliminary Data

| | Mean^{b} (% CV) | | |
|---|---|---|---|
| | | Compound 2 75 mg | % Geometric Least-Squares |
| Compound 2 PK | Compound 2 75 mg | SD + | Mean Ratio^{C} (90% CI) |
| Parameter | SD | Compound 1 40 mg | Compound 1 + Compound 2/ |
| (N = 20) | Alone | BID | Compound 2 alone |
| Cₘₐₓ (μg/mL) | 1.04 (48) | 0.83 (39) | 81.3 (72.5, 91.1) |
| AUC₀₋ₗₐₛₜ (μg * hr/mL) | 8.79 (47) | 7.27 (42) | 83.8 (75.5, 93.2) |
| AUC_{inf} (μg * hr/mL) | 9.46 (46) | 7.92 (42) | 84.9 (76.6, 94.1) |
| T_{1/2} (hr)^{a} | 5.05 (2.69, 7.44) | 6.06 (3.38, 7.90) | N/A |

CV = coefficient of variation,
BID = twice daily,
CI = confidence interval,
N/A = not applicable
^{a}Median (min, max)
^{b}Arithmetic mean
^{c}Geometric least squares means are obtained by the back-transformation of least-squares means of the parameters from an ANOVA using a mixed model based on the natural logarithmic scale. Geometric least squares mean ratio of 100% indicates no change in the parameters.

Based on geometric least-squares mean ratios, Cₘₐₓ, AUC₀₋ₗₐₛₜ, and AUC_{inf} of Compound 2 showed moderate decreases of 19%, 16%, and 15%, respectively, when Compound 2 was coadministered with multiple doses of Compound 1 (40 mg BID) compared with administration of single-dose Compound 2 at 75 mg alone. Median terminal elimination half-lives of Compound 2 were similar between the two treatments.

In summary, the increase in Compound 1 plasma exposure (in terms of AUCs) at 40 and 20 mg is greater with 150 mg BID than 75 mg BID of co-administered Compound 2 (compared with the results shown in Example 5), suggesting a further boosting effect on Compound 1 exposure by Compound 2 at this higher dose. In addition, Compound 1 terminal phase elimination half-lives were prolonged in the presence of Compound 2, suggesting that the large increase in Compound 1 plasma exposure in the presence of Compound 2 appears mainly due to the effect of Compound 2 on the elimination of Compound 1, with a smaller portion of the interaction being due to absorption effects.

### Example 7

### Anti-HCV Activity of the Combination of Compound 1 and Compound 2

This example shows the synergistic interaction between Compound 1 and Compound 2 in an in vitro HCV replication assay.

### Materials and Methods.

Compounds 1 and 2 were synthesized by Gilead Sciences (Foster City, Calif.).

HCV genotype 1b replicon cells (Huh-luc) were obtained from Reblikon (Mainz, Germany). The replicon in these cells is designated I389luc-ubi-neo/NS3-3′/ET and encodes a selectable resistance marker (neomycin phosphotransferase) as well as the firefly luciferase reporter gene. Huh-luc cells were maintained in Dulbecco's Modified Eagle's Medium (DMEM; GIBCO, Carlsbad, Calif.) supplemented with 10% fetal bovine serum (FBS; Hyclone, Logan, Utah) and 0.5 mg/mL of G-418 (GIBCO). Cells were passaged twice a week and maintained at subconfluent levels.

To assess potential advantages of combination anti-HCV therapy, we studied the long-term effects of Compound 1 and Compound 2, alone and in combination, on the HCV replicon in tissue culture. HCV 1b-con replicon cells were passaged in the absence or presence of Compound 1 and Compound 2, individually or in combination, in the absence of neomycin for 20 days. Both compounds were used at concentrations 10 times over their respective EC₅₀ values. In brief, replicon cells (10⁶ cells) were plated in a T75 flask in 15 mL of supplemented DMEM without G418. Compound 1 and/or Compound 2 were added, and the cells were grown in a tissue culture incubator at 37° C., 5% CO₂, to 95% confluency (4 days). At each passage, cells were trypsinized, 10⁶ cells were frozen in 350 mL of RLT RNA lysis buffer (QIAGEN), and an additional 10⁶ cells were seeded into another T75 flask with fresh medium and inhibitors. At the end of the assay, RNA was extracted from frozen replicon cells saved at the time of passage using QIAGEN RNeasy spin kit and was analyzed in a Real-Time RT PCR assay (Applied Biosystems).

### Results.

In untreated control cells, HCV RNA levels were relatively stable over the 6 passages (data not shown). In contrast, the HCV RNA levels were dramatically reduced during treatment with Compound 1 (−2.3 log₁₀ at passage 6) or Compound 2 (−3.1 log₁₀ at passage 6) alone at concentrations 10-fold above their respective EC₅₀ (Table 9). Combination of Compound 1 and Compound 2 displayed an additional 2.1 and 1.3 log₁₀ HCV RNA reduction compared to Compound 1 and Compound 2 alone respectively.

TABLE 9
Replicon RNA log changes at passage 5 and 6 from the baseline
caused by the antiviral treatments

HCV RNA Change
in Log₁₀

| Drug Used in Replicon Treatment | Passage 5 | Passage 6 |
|---|---|---|
| Compound 1 | −2.1 ± 0.5 | −2.4 ± 0.2 |
| Compound 2 | −2.9 ± 0.2 | −3.1 ± 0.4 |
| Compound 1 and Compound 2 | −4.5 ± 0.2 | −4.4 ± 0.02 |

^{a}Values represent the mean ± standard deviation of two independent experiments performed in triplicate

What is claimed:

1. A composition comprising Compound 1 and Compound 2, or salts or solvates of Compound 1 and Compound 2, wherein Compound 1 has the structure shown in Formula 1 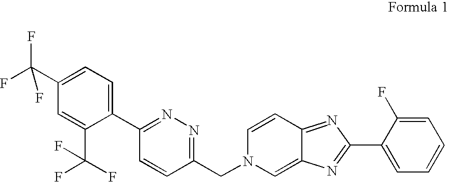
and Compound 2 has the structure shown in Formula 2

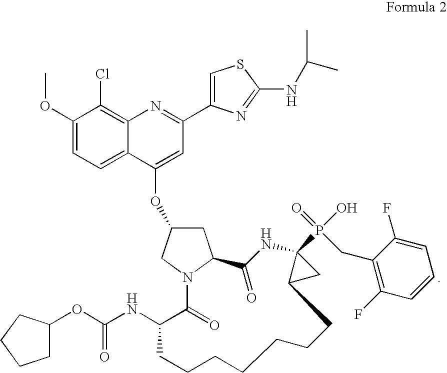

2. The composition of claim 1 wherein Compound 1 is present in an amount of from 1 mg to 100 mg.

3. The composition of claim 1 wherein Compound 1 is present in an amount of from 30 mg to 50 mg.

4. The composition of claim 1 wherein Compound 2 is present in an amount of from 25 mg to 800 mg.

5. The composition of claim 1 wherein Compound 2 is present in an amount of from 100 mg to 400 mg.

6. The composition of claim 1 wherein Compound 1 is present in an amount of from 1 mg to 100 mg, and Compound 2 is present in an amount of from 25 mg to 800 mg.

7. The composition of any one of claims 1 to 6 wherein said composition is a solid composition.

8. The composition of any one of claims 1 to 6 wherein said composition is a liquid composition.

9. The composition of claim 1 further comprising a pharmaceutically acceptable carrier.

10. The composition of claim 9 wherein said composition is a solid composition.

11. The composition of claim 10 wherein said composition is in the form of a tablet.

12. The composition of claim 9 wherein said composition is a liquid composition.

13. The composition of claim 9 wherein Compound 1 is present in an amount of from 1 mg to 100 mg.

14. The composition of claim 9 wherein Compound 1 is present in an amount of from 30 mg to 50 mg.

15. The composition of claim 9 wherein Compound 2 is present in an amount of from 25 mg to 800 mg.

16. The composition of claim 9 wherein Compound 2 is present in an amount of from 100 mg to 400 mg.

17. The composition of claim 9 wherein Compound 1 is present in an amount of from 1 mg to 100 mg, and Compound 2 is present in an amount of from 25 mg to 800 mg.

18. A method for the treatment of HCV infection in a human being, wherein the method comprises the step of administering a therapeutically effective amount of a combination of Compound 1 and Compound 2 to a human being infected with HCV.

19. The method of claim 18 wherein the combination of Compound 1 and Compound 2 comprises a daily dosage of Compound 1 of from 1 mg to 100 mg.

20. The method of claim 18 wherein the combination of Compound 1 and Compound 2 comprises a daily dosage of Compound 1 of from 30 mg to 50 mg.

21. The method of claim 18 wherein the combination of Compound 1 and Compound 2 comprises a daily dosage of Compound 2 of from 25 mg to 800 mg.

22. The method of claim 18 wherein the combination of Compound 1 and Compound 2 comprises a daily dosage of Compound 2 of from 100 mg to 400 mg.

23. The method of claim 18 wherein the combination of Compound 1 and Compound 2 comprises a daily dosage of Compound 1 of from 1 mg to 100 mg and a daily dosage of Compound 2 of from 25 mg to 800 mg.

24. The method of claim 18 wherein Compound 1 is administered to the human being at the same time as Compound 2.

25. The method of claim 18 wherein Compound 1 is administered to the human being before administration of Compound 2 to the human being.

26. The method of claim 18 wherein Compound 1 is administered to the human being after administration of Compound 2 to the human being.

27. The method of claim 24 wherein Compound 1 and Compound 2 are administered to the human being in the form of a tablet.

28. The method of claim 27 wherein the tablet comprises from 1 mg to 100 mg of Compound 1 and from 25 mg to 800 mg of Compound 2.

29. The method of claim 18 wherein Compound 1 and Compound 2 are administered once per day to the human being.

30. The method of claim 18 wherein Compound 1 and Compound 2 are administered more than once per day to the human being.

31. The method of claim 18 wherein Compound 1 and Compound 2 are administered at least once per day to the human being for a period of from 12 weeks to 48 weeks.

32. The method of claim 18 wherein Compound 1 and Compound 2 are administered orally to the human being.

33. The method of claim 18 Wherein Compound 1 and Compound 2 are administered to the human being by injection.

34. The composition of claim 1 wherein Compound 1 is present in an amount of from 20 mg to 40 mg, and Compound 2 is present in an amount of from, 70 mg to 200 mg.

35. The composition of claim 1 wherein Compound 1 is present in an amount of 40 mg and Compound 2 is present in an amount of 75 mg.

36. A composition of claim 34 or claim 35 further comprising a pharmaceutically acceptable excipient.

37. The method of claim 18 wherein the combination of Compound 1 and Compound 2 comprises a daily dosage of Compound 1 of from 20 mg to 40 mg and a daily dosage of Compound 2 of from 70 mg to 200 mg.

38. The method of claim 18 wherein the combination of Compound 1 and Compound 2 comprises a daily dosage of Compound 1 of 40 mg and a daily dosage of Compound 2 of 75 mg.

39. Use of the combination of Compound 1 and Compound 2 in the manufacture of a medicament for the treatment of HCV infection in a human being.

40. A composition comprising Compound 1 and Compound 2 for use in the treatment of HCV infection in a human being.
